(19) United States
(12) Patent Application Publication
(10) Pub. No.: US 2003/0215517 A1
(43) Pub. Date: Nov. 20, 2003

(54) Process for production of crystals of an adjuvant substance, crystals obtained thereby and pharmaceutical preparations containing them

(76) Inventors: Detlef Grawe, Kleinromstedt (DE); Hagen Gerecke, Jena (DE); Peter Hoesel, Jena (DE); Annette Eichardt, Buergel (DE); Sabine Gliesing, Jena (DE); Uwe Mueller, Jena (DE)

Correspondence Address: Michael J. Striker, 103 East Neck Road, Huntington, NY, 11743, US

(21) Appl. No.: 10/417,558

(22) Filed: Apr. 17, 2003

(30) Foreign Application Priority Data
Apr. 23, 2002 (DE) 102 18 110.1

(51) Int. Cl. A61K 9/14

(52) U.S. Cl. 424/489

(57) ABSTRACT

The process for making crystals of an adjuvant substance for pharmaceutical compositions, which have a predetermined average particle size in a predetermined size range and a maximum particle size that does not exceed a predetermined maximum value, includes subjecting a supersaturated solution containing the adjuvant substance to a wet milling by a wet milling apparatus while crystallizing, in order to obtain a primary particle suspension. Crystals obtained according to this process and pharmaceutical preparations containing them are also described.

### BACKGROUND OF THE INVENTION

1. Field of the Invention

The invention relates to a process for production of crystals of an adjuvant substance for a pharmaceutical composition, medicine or ethical drug, whose average particle size is in a predetermined range and whose maximum particle size does not exceed a predetermined value, to the crystals obtained thereby and to the pharmaceutical preparations containing them, especially to low-dosage preparations.

2. Description of the Related Art

Many adjuvant substances are required as microfine powders with predetermined particle size distributions for various pharmaceutical applications. A large-particle-sized or coarse crystallizate with a broad particle size distribution is usually produced by conventional crystallization methods. The final particle size distribution, which is suitable for certain pharmaceutical preparations and dosages, is produced in a special screening and milling process after isolation and drying of crystallizates of this type.

For example, a jet mill can perform a micronizing step in the production of lactose. Average particle sizes of 5 to 10 μm are obtained. An enormous increase in surface area as well as a thermodynamic activation of the surface occurs by partial amorphization and/or by considerable destruction or perturbation of lattice structure. A series of disadvantages are connected with this process, which are described in the literature (Thibert and Tawashhi: “Micronization of Pharmaceutical Solids”, MML Series, Volume 1, Ch. 11, pp. 328-347). An undesirable interaction with the effective ingredient can occur and the adjuvant can be strongly destablized by the partial amorphization. Changes in the particle properties can occur during deposition or in the pharmaceutical composition by recrystallization of the amorphous ingredients. Agglomeration and incrustation or undesirable changes can occur during the milling process. The particle size can be influenced only to a very limited degree by micronization. Lowering the milling pressure of course leads to a slight increase in the average particle size, but also to an undesirable increase in its spread. However a certain minimum pressure is absolutely required for operation of the mill.

Spray-drying (Wendel, et al, “An Overview of Spray-Drying Applications”, Pharmaceutical Technology, Oct 1997, pp. 124-156) is similarly suitable for production of micro-fine particles, however there is a danger of producing unstable amorphous or partially crystalline structures.

It is known from the literature that fine-grained crystals can be produced by precipitation from highly supersaturated solutions or with high stirring speeds. (B. Yu. Shekunov, et al, “Crystallization Process in Pharmaceutical Technology and Drug Delivery Design”, Journal of Crystal Growth 211, pp. 122-136 (2000); Halasz-Peterfi, et al, “Formation of Microparticles of Pharmaceuticals by Homogeneous Nucleation”, Industrial Crystallization, 1999, pp. 1-11).

A suitable method for producing microcrystals by rapidly cooling and intensive mixing is described in U.S. Pat. No. 3,226,389. However these crystallizates often have a large scatter and particle size agglomerates are obtained. Also the desired production of a certain particle size distribution is only possible with difficulty because of the complex interplay of super-saturation, primary and second nuclei formation and crystal growth and/or agglomerate formation.

In EP 0 522 700 the possibility, which is part of the state of the crystallization arts, for providing seed crystals for crystal growth by further defined cooling and heating of a partial flow, which is fed back into the crystallization process is described. With this procedure a grain size increase is obtained in the first place to a grain size largely above 100 μm, in order to improve the filtration and growth processes to obtain a high purity.

The isolation and drying procedures in all the described processes for producing microcrystals in suspensions for low dose preparations can be criticized. It is very difficult to dry fine-grained moist crystallizates, without impairing the grain size distribution.

### SUMMARY OF THE INVENTION

It is an object of the present invention to provide a process for making crystals of an adjuvant substance for a pharmaceutical composition or an ethical drug, which does not have the disadvantages of the known prior art processes and which fulfills the requirements of low-dosage preparations.

According to the invention this object is attained by a process for making crystals of an adjuvant substance for a pharmaceutical composition, whose average grain or particle size is in a predetermined range and whose maximum particle size does not exceed a predetermined value. This process comprises subjecting a supersaturated solution containing the adjuvant substance to a wet milling by means of a wetting milling apparatus while crystallizing, in order to obtain a primary particle suspension.

The term “adjuvant substance”, in the context of the present invention, means a substance contained in a pharmaceutical or medicinal composition in addition to the effective ingredient, which is required so that the effective ingredient can produce the desired activity at the target site in the organism. For example, adjuvant substances of this type, which are used in making drugs, include tablet bursting agents, paste bases, ion exchangers, polymers for depot preparations and aromatizing agents. Lactose and cellulose are representative of the adjuvant substances.

With the process according to the present invention it is surprisingly possible to obtain crystals which are sufficiently stable and which are adjusted in regard to their particle size parameter and thus correct in regard to pharmaceutical requirements for homogeneity of the active ingredient distribution (CUT) and dissolution kinetics for low-dosage formulations. Furthermore the particle size distribution for a certain dosage can be made with a high accuracy and reproducibility. Furthermore the process according to the invention can be performed simply, rapidly and in a cost-effective manner. The crystals can preferably be isolated without impairing their grain size distribution and dried.

### BRIEF DESCRIPTION OF THE DRAWING

The objects, features and advantages of the invention will now be illustrated in more detail with the aid of the following description of the preferred embodiments, with reference to the accompanying figures in which:

FIG. 1 is a graphical illustration of the behavior of the particle size in the course of the crystallization process according to the invention; and

FIG. 2 is another graphical illustration of the behavior of the particle size in the course of the crystallization process according to the invention

### DESCRIPTION OF THE PREFERRED EMBODIMENTS

The average particle size preferably is 1 μm to 25 μm, especially 7 μm to 15 μm, entirely especially 5 μm to 10 μm. The maximum particle size preferably does not exceed 100 μm, more preferably 80 μm. The “maximum particle size” means that no particle has a size that is greater than the stated value. Within these limits for the average particle size and the maximum particle size the particle size distribution is selected in a beneficial way so that the pharmaceutical specifications regarding CUT and dissolution kinetics correspond to those for low-dose formulations.

In the process according to the invention a supersaturated solution of the adjuvant substance is used. The solution contains the adjuvant as a solute, which is dissolved for that purpose in a solvent. The term “solvent” is understood to encompass mixtures of different solvents. A supersaturated solution used in the process according to the invention contains more dissolved material than it would when the solution is in thermodynamic equilibrium. Supersaturated solutions, in which crystal nuclei spontaneously form, can be used in the process according to the invention. Methods of making supersaturated solutions of adjuvant substances are known to those skilled in the art.

In a preferred embodiment of the process according to the invention the supersaturated solution contains from 1 percent by weight to 60 percent by weight, preferably 5 percent by weight to 35 percent by weight, of the adjuvant, in relation to the supersaturated solution. The above-described advantages of the process according to the invention can be achieved in an especially beneficial manner with these supersaturated solutions.

The crystallization is performed in a vessel, which is equipped with a stirrer. For example, the crystallization vessel can be equipped for that purpose.

In the process according to the invention wet milling is performed by a wet milling apparatus during crystallization. The crystallization can proceed from the saturated solution, when the wet milling has been started. Suitable apparatuses for wet milling include dispersion tools and homogenizers, such as rotor-stator apparatuses, stirring mills, roller mills and colloid mills.

The making of crystals according to the invention occurs, as described above, by crystallization from a solvent or solvent mixture, which is supersatured with the adjuvant substance. During crystallizing wet milling by a wet milling apparatus, especially a rotor-stator apparatus or a colloid mill, is performed. The wet milling is performed either shortly after crystallization has begun or before it has begun. The apparatus for wet milling can be used immediately as an additional stirring device in the crystallization vessel or in a by-pass loop that goes around the crystallization vessel. The use of the by-pass loop is especially beneficial, since the apparatus is used at the same time as a supply unit. If a rotor-stator apparatus is used, the peripheral rotation speed can be 10 m/s to 50 m/s, preferably 20 m/s to 40 m/s. A very high secondary nuclei formation rate is produced by the additional energy input caused by the wet milling, especially by the rotor-stator apparatus. The individual crystal growth is greatly reduced because of that energy input. Also the inevitably formed agglomerates are broken up in narrow gaps. Thus a fine primary particle size is the result, whose average particle size is between 1 μm and 5 μm and whose maximum particle size is not greater than 25 μm to 80 μm. These particle parameters can already be sufficient for low dose formulations.

In order to be able to make crystals that meet the pharmaceutical requirements, even for larger particle sizes, with a definite particle size distribution with suitable accuracy and better reproducibility, the primary suspension is preferably subjected to an oscillatory temperature profile. For that purpose the fine primary suspension produced is heated to a temperature Tₘₐₓ below the solubility limit of the primary particles in the suspension and subsequently cooled slowly to a temperature Tₘᵢₙ, which is above the freezing point of the suspension. On heating the fine-grained fraction of the primary particle suspension is dissolved and precipitated on the particle size fraction present during the cooling process. Because of that a definite shift in the particle size distribution to the larger range occurs. Preferably Tₘₐₓ is selected so that between 10 and 95, preferably 20 to 50 and more preferably about 30, percent by weight of the primary particles dissolve during the heating. The fraction of dissolved primary particles is selected according to the predetermined grain size, which again is determined by the type of low-dosage formulation. If a higher proportion of the primary particles dissolve, larger-sized particles result.

In a preferred embodiment of the process according to the invention Tₘᵢₙ is selected so that the dissolved primary particles substantially re-crystallize again. If it is particularly desirable to reduce the losses of effective ingredient, nearly all of the dissolved primary particles are re-crystallized on the still remaining primary particles.

It is especially preferable when the cooling from Tₘₐₓ to Tₘᵢₙ occurs during 1 min to 10 hours, especially during 0.5 hours to 2 hours.

The cooling side of the temperature profile should be controlled so that the fresh nuclei formation is kept as small as possible. The size of this coarsening depends on the amount of the crystallizates dissolved in the heating cycle, which again is determined by the position of both temperatures Tₘₐₓ and Tₘᵢₙ in relation to the solubility limit and the solid concentration of the suspension. This heating-cooling cycle can be repeated often, preferably 1 to 20 times, until the desired particle size distribution is obtained. The controlling parameters are thus Tₘₐₓ, Tₘᵢₙ and the number of cycles. The more the desired coarsening, the less Tₘₐₓ should be. Thus one can approach the desired final particle size with small steps. The development of the dissolved portion of the crystallizate in the heating periods is thus dimensioned so that the maximum particle diameter increases still only to a very small extent and the coarsening occurs in the region of the fine particles.

After passing through the oscillatory temperature profile the obtained crystal suspension can be filtered and washed with a solvent, when the active ingredient is only soluble to a small extent, e.g. less than 1 percent by weight. For example, these solvents are methyl-t.-butyl ether(MTBE), hexane, heptane, water or mixtures of two or more of these solvents. Thus in subsequent drying processes, which occur preferably by a drying gas or in vacuum directly in the filtration unit, bridge formation and agglomeration of the particles are avoided.

The drying can occur by convection or vacuum drying in a stirred or moving bed.

When a conventional filtration and drying is difficult and leads to impairment of the particle size distribution produced during the crystallization, for example in the case of very fine particle sizes, alternatively the filtered and washed filter cake is suspended with a suspending liquid. The suspending liquid should be a liquid, preferably water, in which the adjuvant is only slightly soluble, for example less than one percent by weight. The obtained suspension can be converted into the dried solid form of the adjuvant substance by spray drying.

The subject matter of the invention also includes crystals of the adjuvant substance, which are obtained by the above-described process according to the invention. To perform the process in the above-described manner, the detailed description of the process here is referred to.

The subject matter of the invention also includes pharmaceutical formulations or preparations, which contain the crystals of the adjuvant substance obtained according to the process of the invention.

An essential result of the invention is that microcrystals of the adjuvant substance are obtained which are considerably more stable than the micronizate that is currently known, since they first have a reduced specific surface area and second they have a unperturbed and highly crystalline surface area because of the crystallization process according to the invention.

A further result is that the particle size distribution and solubility properties of the adjuvant crystals prepared by the process according to the invention meet the specifications for pharmaceutical compositions, medicines and drugs regarding CUT and dissolution.

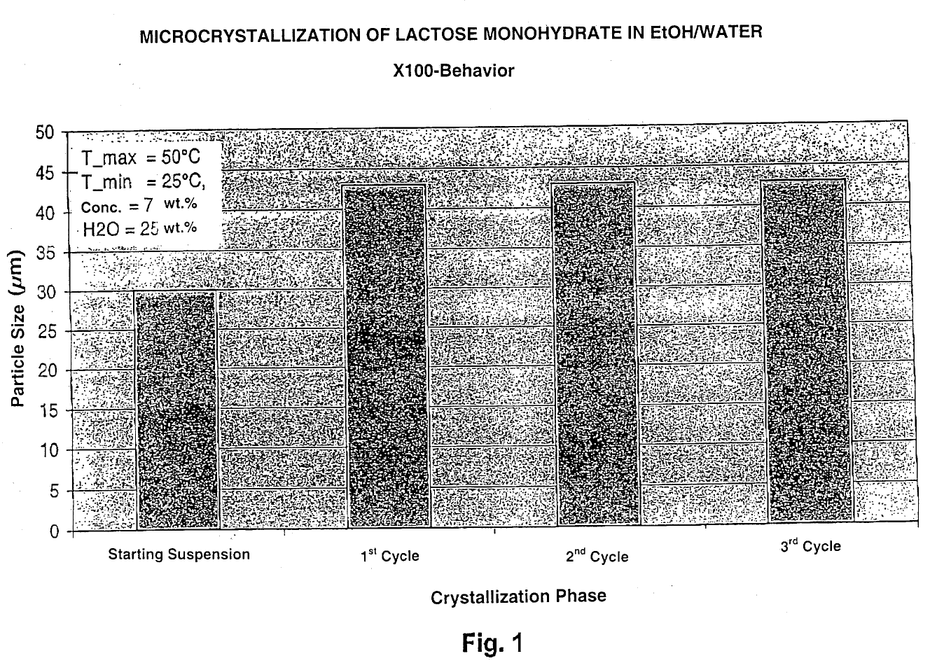

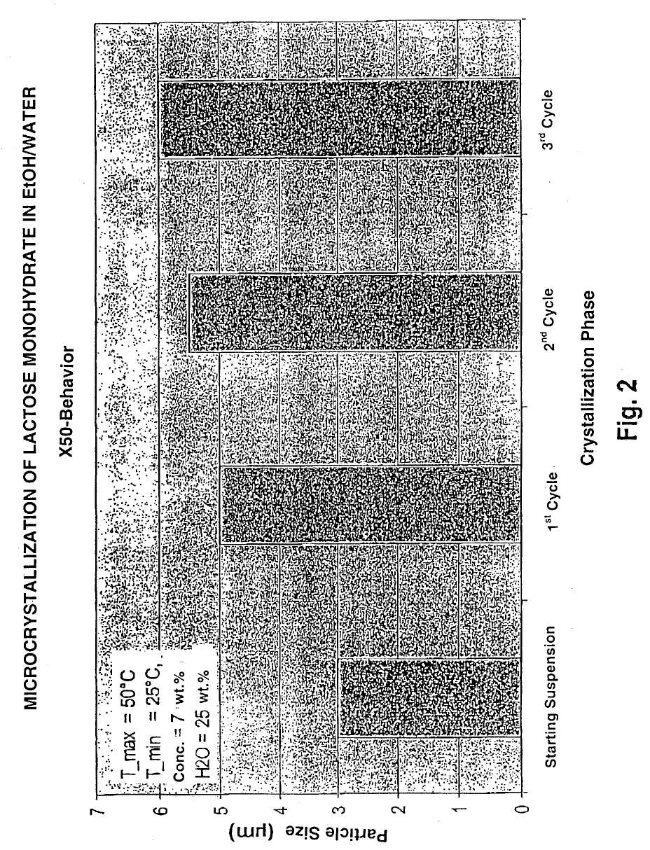

A further important result is that the pharmaceutically required particle size distribution of the adjuvant substance can be produced with higher reproducibility and accuracy with the process according to the invention. In FIGS. 1 and 2 the development of the grain size or particle size in the crystallization process is illustrated. The scatter of the particle size distribution is clearly reduced and the maximum grain size is clearly only slightly increased in spite a multiple increase in the average particle size. This assists in attaining good CUT values, also for low-dosage formulations.

Furthermore the grain size distribution produced in the suspension also is maintained in the dried solid body.

TABLE I
LACTOSE-MONOHYDRATE:
PARTICLE SIZE DISTRIBUTION
BEFORE AND AFTER DRYING

| | X10 | X50 | X100 |
|---|---|---|---|
| Suspension* | 2.11*** | 5.9 | 43 |
| After spray-drying** | 2.25 | 6.0 | 43 |

*7 percent by weight lactose monohydrate in EtOH/water, 75/25 (w/w)
**dry powder
***particle diameters in μm

The particle size distribution in Table I was measured with a Sympatec HELOS (H0445), dry dispersion system (RODOS), pressure 2 bar.

The following examples serve to illustrate the invention, but do not limit the broad concept of the invention expressed generally above or in the claims appended below.

### EXAMPLES

### Example 1

In a sulfonation flask with a blade mixer and a heating/cooling bath 30 g of lactose monohydrate are dissolved in 100 g of water at 62° C. A rotor-stator dispersing apparatus (Ultra Turrax, T25 basic, with S25N-25F) is operated with a rotation speed of 12000 to 22000 rpm to prepare the solution. Then 300 g of ethanol are added. After 1 to 2 minutes crystallization begins at 25 to 35° C. The Ultra Turrax is operated for an additional 10 minutes and then is shut off.

The starting suspension obtained is heated at 50° C. and subsequently cooled within an interval of 1 hour at 20° C. This procedure is repeated still twice more.

Particle Size Development

| | X50, μm | X100, μm |
|---|---|---|
| Starting Suspension | 3 | 30 |
| 1^{st} Cycle | 5 | 43 |
| 2^{nd} Cycle | 5.5 | 43 |
| 3^{rd} Cycle | 6 | 43 |

### Example 2

Subsequently the suspension obtained in example 1 is filtered by means of a frit and washed with 100 ml of ethanol. The filter cake is suspended in 300 ml ethanol. The suspension is spray-dried under the following conditions in a laboratory spray-drier with two nozzles (2 mm) (QVF/Yamato):

| | | |
|---|---|---|
| Drying gas entrance temperature: | 170° | C. |
| Drying gas exit temperature: | 60° | C. |
| Drying gas throughput: | 23 | m³/min |
| Spray nozzle (d = 2 mm) | 2.5 | bar |
| Feed: | 8 to 10 | ml/min |

Microcrystals are obtained in a separating filter of the spray-drier with the following particle size distribution:

| | Particle size (μm) |
|---|---|
| X10 | 2.5 |
| X50 | 5.9 |
| X100 | 43 |
| Moisture content: | 4.5% |

The disclosure in German Patent Application 102 18 110.4 of Apr. 23, 2002 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in a process for production of crystals of an adjuvant substance for a pharmaceutical composition, crystals obtained thereby and pharmaceutical preparations containing them, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A process for making crystals of an adjuvant substance for a pharmaceutical composition, said crystals having an average particle size in a predetermined size range and a maximum particle size that does not exceed a predetermined maximum value, said process comprising subjecting a supersaturated solution containing said adjuvant substance to a wet milling by a wet milling apparatus while crystallizing, in order to obtain a primary particle suspension.

2. The process as defined in claim 1, wherein said average particle size of said crystals of said adjuvant substance is from 1 μm to 25 μm.

3. The process as defined in claim 1, wherein said predetermined maximum value is 100 μm.

4. The process as defined in claim 1, wherein said supersaturated solution contains from 1 to 60 percent by weight of said adjuvant substance, based on said supersaturated solution.

5. The process as defined in claim 1, further comprising preparing said supersaturated solution by dissolving said adjuvant substance in a solvent at a temperature below a boiling point of said solvent to form a resulting solution and subsequently cooling said resulting solution to a temperature above a freezing point of the resulting solution.

6. The process as defined in claim 1, wherein said crystallizing is performed in a vessel or container having a stirring device.

7. The process as defined in claim 1, wherein said wet milling apparatus is a rotor-stator apparatus, a stirring mill, a roller mill or a colloid mill.

8. The process as defined in claim 1, further comprising heating said primary particle suspension at a temperature (Tₘₐₓ) below a solubility limit of primary particles of the primary particle suspension and subsequently cooling to a temperature above a freezing point (Tₘᵢₙ) of the primary particle suspension.

9. The process as defined in claim 8, wherein said supersaturated solution comprises a solvent and said temperature (Tₘₐₓ) below said solubility limit is selected so that from 10 to 95 percent by weight of said primary particles dissolve in said solvent.

10. The process as defined in claim 9, wherein said temperature above said freezing point (Tₘᵢₙ) is selected so that dissolved primary particles are substantially re-crystallized.

11. The process as defined in claim 10, wherein said cooling from said temperature (Tₘₐₓ) below said solubility limit to said temperature above said freezing point (Tₘᵢₙ) occurs during a time interval of 1 minute to 10 hours.

12. The process as defined in claim 8, wherein said heating at said temperature (Tₘₐₓ) below said solubility limit and said cooling at said temperature above said freezing point are performed from 1 to 20 times.

13. Crystals of an adjuvant substance, said crystals having an average particle size in a predetermined size range and a maximum particle size that does not exceed a predetermined maximum value, wherein said crystals are made by a process comprising subjecting a supersaturated solution containing said adjuvant substance to a wet milling by a wet milling apparatus while crystallizing, in order to obtain a primary particle suspension.

14. The crystals as defined in claim 13, wherein said average particle size is from 1 μm to 25 μm and said predetermined maximum value is 100 μm.

15. The crystals as defined in claim 13, wherein said supersaturated solution contains from 1 to 60 percent by weight of said adjuvant substance, based on said supersaturated solution, and said process comprises preparing said supersaturated solution by dissolving said adjuvant substance in a solvent at a temperature below a boiling point of said solvent to form a resulting solution and subsequently cooling said resulting solution to a temperature above a freezing point of the resulting solution.

16. The crystals as defined in claim 13, wherein said supersaturated solution comprises a solvent; said process comprises heating said primary particle suspension at a temperature (Tₘₐₓ) below a solubility limit of primary particles of the primary particle suspension and subsequently cooling to a temperature above a freezing point (Tₘᵢₙ) of the primary particle suspension and wherein said temperature (Tₘₐₓ) below said solubility limit is selected so that from 10 to 95 percent by weight of said primary particles dissolve in said solvent and said temperature above said freezing point (Tₘᵢₙ) is selected so that dissolved primary particles are substantially re-crystallized, said cooling from said temperature (Tₘₐₓ) below said solubility limit to said temperature above said freezing point (Tₘᵢₙ) occurs during a time interval of 1 minute to 10 hours.

17. The crystals as defined in claim 13, wherein said crystallizing is performed in a vessel or container having a stirring device and said wet milling apparatus is a rotor-stator apparatus, a stirring mill, a roller mill or a colloid mill.

18. A pharmaceutical preparation containing crystals of an adjuvant substance, said crystals having an average particle size and a maximum particle size, wherein said crystals are made by a process comprising subjecting a supersaturated solution containing the adjuvant substance to a wet milling by a wet milling apparatus while crystallizing, in order to obtain a primary particle suspension.

19. The pharmaceutical preparation as defined in claim 18, wherein said average particle size is from 1 μm to 25 μm and said maximum particle size is 100 μm.

20. The pharmaceutical preparation as defined in claim 18, wherein said crystallizing is performed in a vessel or container having a stirring device and said wet milling apparatus is a rotor-stator apparatus, a stirring mill, a roller mill or a colloid mill.

21. The pharmaceutical preparation as defined in claim 18, wherein said supersaturated solution contains from 1 to 60 percent by weight of said adjuvant substance, based on said supersaturated solution, and said process comprises preparing said supersaturated solution by dissolving said adjuvant substance in a solvent at a temperature below a boiling point of said solvent to form a resulting solution and subsequently cooling said resulting solution to a temperature above a freezing point of the resulting solution.
